(19) United States
(12) Patent Application Publication
(10) Pub. No.: US 2024/0303826 A1
(43) Pub. Date: Sep. 12, 2024

(54) IMAGE PROCESSING APPARATUS, IMAGE PROCESSING METHOD, AND RECORDING MEDIUM

(71) Applicant: Konica Minolta, Inc., Tokyo (JP)

(72) Inventors: Hiromu OHARA, Tokyo (JP); Takuya YAMAMURA, Hachioji-shi (JP)

(21) Appl. No.: 18/583,046

(22) Filed: Feb. 21, 2024

(30) Foreign Application Priority Data
Mar. 9, 2023 (JP) 2023-036088

(51) Int. Cl. G06T 7/20; G06T 5/20; G06T 7/13; G06V 10/25

(52) U.S. Cl. CPC G06T 7/20; G06T 5/20; G06T 7/13; G06V 10/25

(57) ABSTRACT

An image processing apparatus includes a hardware processor that acquires a plurality of frame images constituting a radiographed dynamic image of a subject, sets an analysis portion in a reference frame image among the plurality of frame images, selects one tracking algorithm from a plurality of tracking algorithms, tracks the analysis portion in a time direction on the basis of the one tracking algorithm, and outputs a tracking result of the tracking.

### CROSS-REFERENCE TO RELATED APPLICATIONS

The entire disclosure of Japanese Patent Application No. 2023-036088 filed on Mar. 9, 2023 is incorporated herein by reference in its entirety.

### BACKGROUND OF THE INVENTION

### Technical Field

The present invention relates to an image processing apparatus, an image processing method, and a recording medium.

### Description of Related Art

In recent years, an image processing apparatus has been known which acquires a captured dynamic image of motion of a target site of a living body through radiographing and performs image processing. The image processing apparatus tracks a region of interest set from a plurality of captured frame images and performs motion estimation processing.

In tracking of dynamic images, a plurality of analysis algorithms are used. Each of the plurality of analysis algorithms has advantages and disadvantages depending on the content. It is therefore necessary to select an appropriate analysis algorithm as appropriate in accordance with an analysis target, a situation, and the like.

Thus, for example, JP 2021-194164 A discloses an image processing apparatus that executes a plurality of kinds of motion estimation processing on a plurality of pieces of medical image data and selects motion information with a high likelihood among a plurality of pieces of motion information.

However, in the invention in JP 2021-194164 A, the plurality of kinds of motion estimation processing determined in advance are performed without an analysis target and a situation are taken into account. Thus, motion estimation processing which is not originally necessary is executed, which results in taking time to obtain an analysis result.

### SUMMARY OF THE INVENTION

The present invention has been made in view of such circumstances. An object of the present invention is to provide an image processing apparatus, an image processing method, and a recording medium capable of acquiring a desired analysis result more quickly.

To achieve at least one of the abovementioned objects, according to an aspect of the present invention, an image processing apparatus reflecting one aspect of the present invention includes a hardware processor that:
acquires a plurality of frame images constituting a radiographed dynamic image of a subject,
sets an analysis portion in a reference frame image among the plurality of frame images,
selects one tracking algorithm from a plurality of tracking algorithms,
tracks the analysis portion in a time direction on the basis of the one tracking algorithm, and
outputs a tracking result of the tracking.

To achieve at least one of the abovementioned objects, according to an aspect of the present invention, an image processing method of an image processing apparatus reflecting one aspect of the present invention includes:
inputting a plurality of frame images constituting a radiographed dynamic image of a subject,
setting an analysis portion in a reference frame image among the frame images,
selecting one tracking algorithm from a plurality of tracking algorithms,
tracking the analysis portion in a time direction on the basis of the one tracking algorithm, and
outputting a tracking result of the tracking.

To achieve at least one of the abovementioned objects, according to an aspect of the present invention, a non-transitory computer readable recording medium reflecting one aspect of the present invention stores:
a program for causing a computer of an image processing apparatus to perform:
acquiring a plurality of frame images constituting a radiographed dynamic image of a subject,
setting an analysis portion in a reference frame image among the plurality of frame images,
selecting one tracking algorithm from a plurality of tracking algorithms,
tracking the analysis portion in a time direction on the basis of the one tracking algorithm, and
outputting a tracking result of the tracking.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram of a radiographic imaging system;

FIG. 2 is a block diagram of an image processing apparatus;

FIG. 3 is a flowchart of dynamic image analysis processing by the image processing apparatus;

FIG. 4 is a view illustrating an example of a startup screen of an image processing tool;

FIG. 5 is a view illustrating an example of the image processing tool in which a selected dynamic image is displayed;

FIG. 6 is a view illustrating an example of an analysis condition setting screen of the image processing tool;

FIG. 7 is a view illustrating an example of a frame image in which an ROI is set;

FIG. 8 is a view illustrating an example of analysis content in a case where the shoulder joint is set as the ROI;

FIG. 9 is a view illustrating an example of analysis content in a case where the vertebral body is set as the ROI;

FIG. 10A is a view illustrating an example of analysis content in a case where the chest region is set as the ROI;

FIG. 10B is a view illustrating an example of analysis content in a case where the chest region is set as the ROI;

FIG. 11A is a view illustrating a tracking result of the knee region in a case where positioning is not performed;

FIG. 11B is a view illustrating a tracking result of the knee region in a case where positioning is performed; and

FIG. 12 is a view illustrating an example of a frame image in which tracking lines that are a tracking result is displayed.

### DETAILED DESCRIPTION

Hereinafter, one or more embodiments of the present invention will be described with reference to the drawings. However, the scope of the invention is not limited to the disclosed embodiments.

### Configuration of Radiographic Imaging System

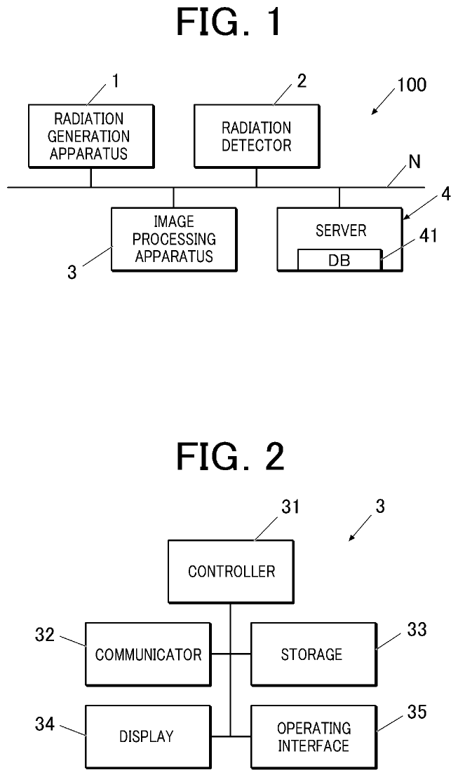

First, a schematic configuration of a radiographic imaging system 100 according to the present embodiment will be described. FIG. 1 is a block diagram illustrating the radiographic imaging system 100.

As illustrated in FIG. 1, the radiographic imaging system 100 of the present embodiment includes a radiation generation apparatus 1, a radiation detector 2, an image processing apparatus 3, and a server 4.

These apparatuses can perform communication with each other via a communication network N. Further, the respective apparatuses constituting the radiographic imaging system 100 conform to digital image and communication in medium (DICOM) standards. Thus, the respective apparatuses perform communication in conformity with DICOM.

Note that the radiographic imaging system 100 may be connected to a host system. The host system in the present invention is, for example, a hospital information system (HIS), a radiology information system (RIS), a picture archiving and communication system (PACS), or the like.

### Radiation Generation Apparatus

The radiation generation apparatus 1 includes a generator, a radiation source, and the like. The generator applies a voltage in accordance with radiation irradiation conditions set in advance such as a tube voltage, a tube current and an irradiation period (mAs value) on the basis of an irradiation instruction of the radiation generation apparatus 1. The radiation source generates radiation such as, for example, an X-ray of an amount in accordance with the applied voltage when the voltage is generated from the generator. Further, the radiation generation apparatus 1 generates radiation in an aspect in accordance with a dynamic image to be radiographed.

Note that the radiation generation apparatus 1 may be installed in a radiographing room. Alternatively, the radiation generation apparatus 1 may be configured to be able to move in a visiting car with the image processing apparatus 3, and the like.

### Radiation Detector

The radiation detector 2 is constituted with a semiconductor image sensor such as a flat panel display (FPD). The radiation detector 2 includes a radiation detection element, a substrate, a scanning circuit, a readout circuit, a controller, an output device, and the like. The radiation detection element generates charges in accordance with a radiation dose by receiving radiation. The substrate includes switching elements that accumulate and discharge charges, and pixels are arranged two-dimensionally, that is, in a matrix. The scanning circuit switches ON and OFF of each switching element. The readout circuit reads out an amount of the charges discharged from each pixel as a signal value. The controller generates a frame image from a plurality of signal values read out by the readout circuit. The output device outputs data, and the like, of the frame image generated by the controller to outside.

Then, the radiation detector 2 generates a dynamic image including a plurality of frame images in accordance with radiated radiation in synchronization with a timing at which radiation is radiated from the radiation generation apparatus 1.

Here, kymography includes radiographing of a moving image, but does not include radiographing of a still image while displaying a moving image. Further, a dynamic image includes a moving image, but does not include an image obtained by radiographing a still image while displaying a moving image.

While a case has been described above as an example where the radiation detector 2 is a so-called direct type, the present invention is not limited to this. In other words, the radiation detector 2 may be a so-called indirect type. The indirect type radiation detector 2 includes a built-in scintillator that converts radiated radiation into light of other wavelengths such as visible light. The indirect type radiation detector 2 generates charges in accordance with the light converted at the scintillator.

Further, the radiation detector 2 may be a dedicated type integrated with a radiographic stand or a portable type, particularly, a cassette type.

### Image Processing Apparatus

The image processing apparatus 3 includes a personal computer (PC), a dedicated apparatus, or the like. The image processing apparatus 3 executes dynamic image analysis processing on the plurality of frame images acquired from the radiation detector 2. The image processing apparatus 3 will be described in detail later.

Note that the image processing apparatus 3 may be a console that can set various kinds of radiographing conditions at the radiation generation apparatus 1, the radiation detector 2, and the like. Various kinds of radiographing conditions are, for example, a tube voltage, a tube current, an irradiation period (mAs value), a frame rate, a body type of the subject, whether or not there is a grid, and the like. Further, setting of the various kinds of radiographing conditions is based on radiographing order information acquired from other systems such as the HIS and the RIS or operation by a user.

### Server

The server 4 includes a PC, a dedicated apparatus, or a virtual server on the cloud, or the like. The server 4 includes a database (DB) 41. The DB 41 can accumulate radiographed dynamic images generated by the radiation detector 2 and processing results of the image processing apparatus 3.

Note that while in the present embodiment, a case has been described as an example where the DB 41 is provided in the server 4 independent of the image processing apparatus 3, and the like, the present invention is not limited to this. The DB 41 may be provided in the image processing apparatus 3. Further, the DB 41 may be provided in another apparatus included in the radiographic imaging system 100.

Still further, for example, in a case where another system such as a PACS is connected to the radiographic imaging system 100, the DB 41 may be provided in the other system.

In the radiographic imaging system 100, the radiation source of the radiation generation apparatus 1 and the radiation detector 2 are provided to face each other across the subject. Further, if the subject is irradiated with radiation from the radiation source, the subject can be radiographed.

The radiographic imaging system 100 according to the present embodiment repeats operation of irradiation with pulsed radiation a plurality of times (for example, 15 times/second) in a short period of time from the radiation source with one time of radiographing operation. Further, the radiation detector 2 generates a dynamic image of the subject by repeating generation of a dynamic image a plurality of times in a short period of time in accordance with the irradiation.

### Configuration of Image Processing Apparatus

Next, a specific configuration of the image processing apparatus 3 included in the radiographic imaging system 100 will be described. FIG. 2 is a block diagram illustrating the image processing apparatus 3.

As illustrated in FIG. 2, the image processing apparatus 3 according to the present embodiment includes a controller 31 (i.e., hardware processor), a communicator 32, a storage 33, a display 34, an operating interface 35, and the like. These components are electrically connected with a bus, or the like.

The controller 31 includes a central processing unit (CPU), a random access memory (RAM), and the like. The CPU of the controller 31 reads out various kinds of programs stored in the storage 33 and loads the programs in the RAM. Then, the CPU executes various kinds of processing in accordance with the programs loaded in the RAM and performs centralized control of operation of the respective components of the image processing apparatus 3.

The communicator 32 includes a communication module, and the like. The image processing apparatus 3 and other apparatuses are connected via the communication network N such as a local area network (LAN), a wide area network (WAN) and the Internet. The communicator 32 transmits/receives various kinds of signals and various kinds of data with other apparatuses connected via the communication network N.

The storage 33 includes a non-volatile semiconductor memory, a hard disk, and the like. The storage 33 stores various kinds of programs to be executed by the controller 31 and parameters, and the like, necessary for execution of the programs. The storage 33 stores various kinds of programs related to an image processing tool that executes dynamic image analysis processing which will be described later. The storage 33 may be able to store a dynamic image.

The display 34 includes a liquid crystal display (LCD), a cathode ray tube (CRT), and the like. Further, the display 34 displays a radiographed dynamic image, a measurement result, and the like, on the basis of a control signal input from the controller 31.

The operating interface 35 includes a keyboard including various kinds of function keys, a pointing device such as a mouse, a touch panel laminated on a surface of a display device, and the like.

The operating interface 35 outputs a control signal in accordance with operation of the user to the controller 31.

Note that a display device such as a tablet terminal including a display and an operating interface may be connected to the image processing apparatus 3. In a case where the display device is connected to the image processing apparatus 3, the display 34 and the operating interface 35 do not have to be provided in the image processing apparatus 3.

### Dynamic Image Analysis Processing

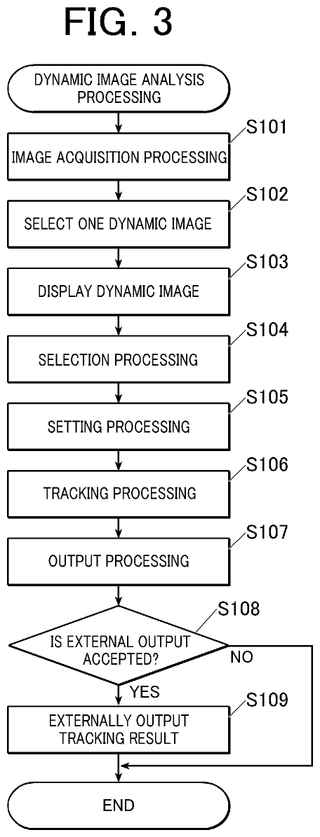

The controller 31 of the image processing apparatus 3 executes dynamic image analysis processing as illustrated in FIG. 3 by being triggered by predetermined conditions being satisfied. The predetermined conditions include, for example, starting of power supply of the image processing apparatus 3, predetermined operation on the operating interface 35, acquisition of data of the dynamic images or a control signal by the image processing apparatus 3, and the like.

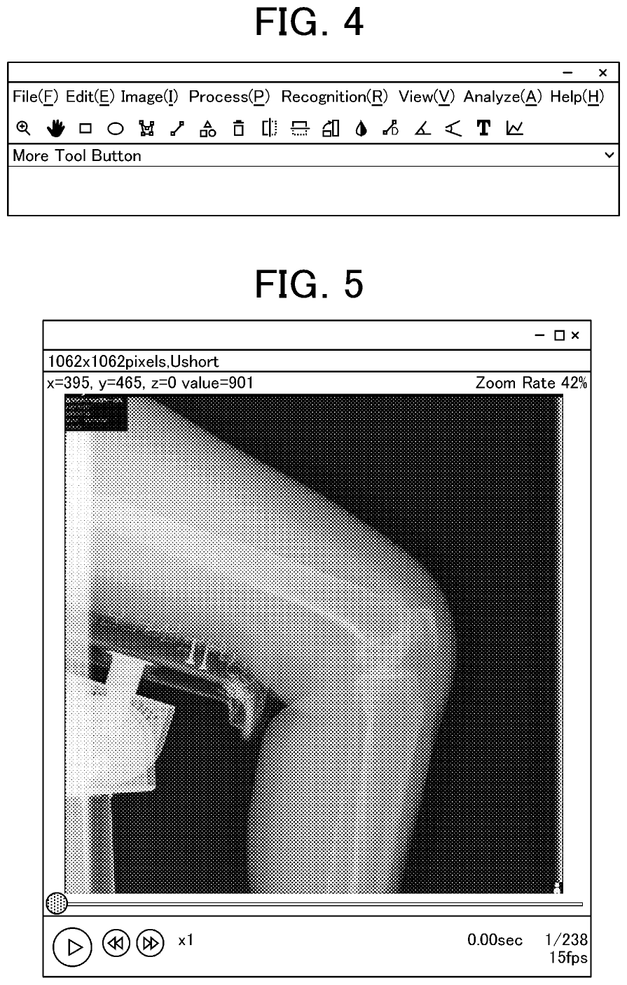

FIG. 4 illustrates an example of a startup screen of the image processing tool according to the present embodiment.

The controller 31 first executes image acquisition processing (step S101). In the image acquisition processing, dynamic images are received from other apparatuses via the communicator 32. The other apparatuses include the radiation detector 2, the PACS, and the like. Note that while individual frame images may be sequentially acquired when the dynamic images are received, it is preferable to collectively acquire all the frame images.

The controller 31 functions as an acquirer by executing the image acquisition processing as described above.

Note that the image acquisition processing may be executed by loading the dynamic images stored in a storage medium without interposition of the communicator 32. Further, in a case where the dynamic image analysis processing is started by being triggered by acquisition of data of the dynamic images from other apparatuses, the image acquisition processing is not required.

The user selects one dynamic image from the dynamic images acquired by the controller 31 in the image acquisition processing (step S102).

If the one dynamic image is selected, the controller 31 causes the selected dynamic image to be displayed at the display 34 as illustrated in FIG. 5 (step S103).

The user selects analysis conditions of the selected one dynamic image (step S104).

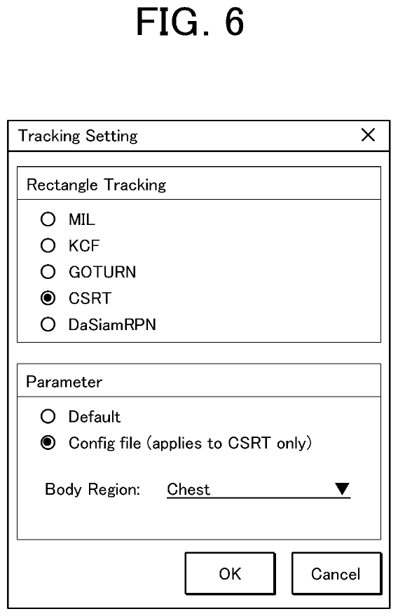

FIG. 6 illustrates an example of an analysis condition setting screen of the dynamic image. As illustrated in FIG. 6, in the present step, for example, which tracking algorithm is to be used can be selected.

The controller 31 functions as a selector by executing selection processing of accepting selection as to which tracking algorithm is to be used.

Note that in step S104, parameters that can be changed in the selected tracking algorithm can be set. The parameters that can be selected in step S104 are, for example, filter_lr, template_size, maxLevel, and the like. In this manner, by the user setting parameters optimal for the analysis target, analysis can be performed with higher accuracy.

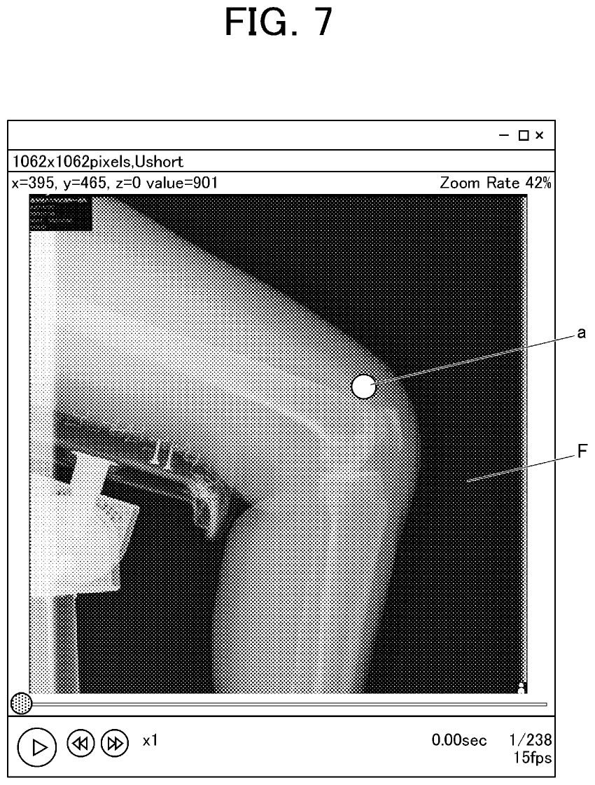

After the analysis conditions are set, as illustrated in FIG. 7, the user sets at least one region of interest (ROI) a that is an analysis portion on one frame image constituting the dynamic image (step S105).

The controller 31 functions as a setter by executing setting processing of setting the ROIa on the frame image. Further, in the following description, the frame image on which the ROIa is set in one dynamic image will be set as a reference frame image F.

Note that while in FIG. 7, a case has been described as an example where the reference frame image F is a first frame image among the plurality of frame images, the present invention is not limited to this. The reference frame image F may be any frame image of the one dynamic image. In this manner, by allowing the ROIa to be set in a desired frame image, the user can more easily set the ROIa.

It is only necessary to set at least one ROIa in the reference frame image F, and a plurality of ROIa may be set. For example, in a case where two ROIa are set in the reference frame image F, motion of the both points may be independently tracked, or a distance between the both points may be acquired. Further, for example, in a case where three ROIa are set in the reference image frame image F, an angle formed by two lines obtained from three points may be acquired. Further, for example, in a case where four ROIa are set in the reference frame image F, a distance or an angle of two lines may be acquired. In particular, in a case where it is desired to grasp rotation (twist) or lateral displacement (angle change) of the target site, measurement accuracy is improved by setting a plurality of ROIa in the reference frame image F.

Further, while in the above description, a case has been described as an example where the user manually sets the ROIa, the present invention is not limited to this. For example, a key for setting the ROIa in a predetermined portion, such as a “vertex of right diaphragm” may be provided. In a case where the user operates the key, the controller 31 automatically detects the predetermined portion from the frame image and sets the portion as the ROIa. Then, the controller 31 executes analysis in accordance with the predetermined portion.

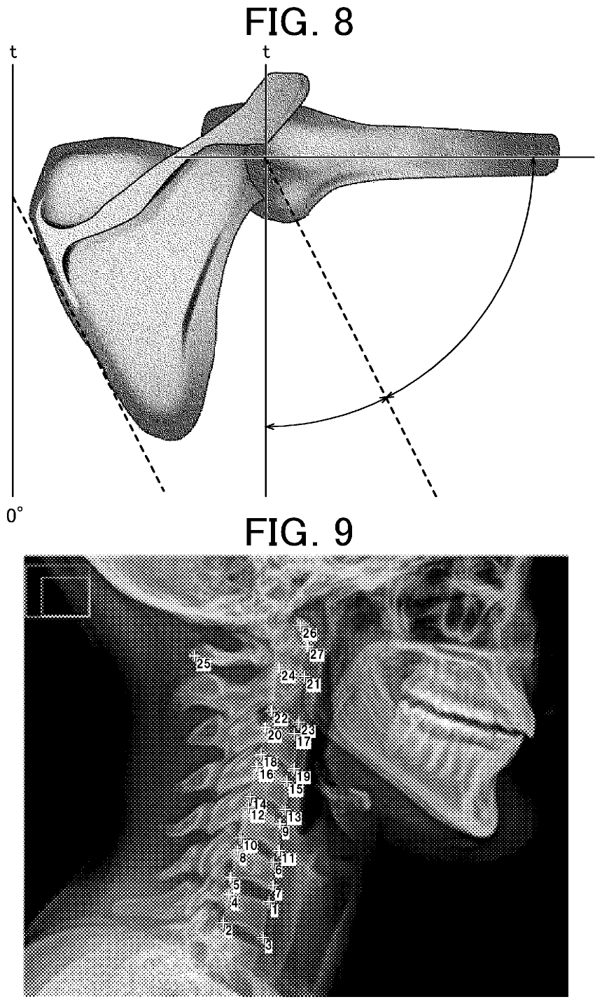

For example, in a case where the predetermined portion is the shoulder joint, the controller 31 detects the shoulder blade and the humerus in each frame image of the selected one dynamic image. Then, as illustrated in FIG. 8, the controller 31 automatically analyzes angles formed by a vertical line t and the shoulder blade and the humerus.

Further, in a case where the predetermined portion is the vertebral body such as the cervical vertebra and the lumber vertebra, the controller 31 detects the vertebral body in each frame image of the selected one dynamic image. Further, as illustrated in FIG. 9, the controller 31 automatically analyzes positions of four corners or a center position of each vertebra. Note that in step S109 which will be described later, in a case where a dynamic analysis result of the vertebra is output, it is preferable to cause the controller 31 to output an analysis result standardized at a position of a bone C7 at a lowest end.

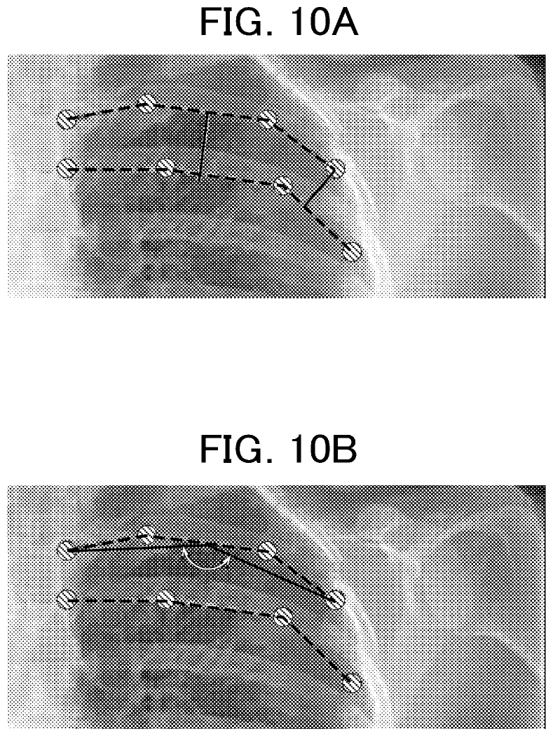

Alternatively, in a case where the predetermined portion is the chest region such as the rib, the controller 31 detects the rib in each frame image of the selected one dynamic image. Further, the controller 31 sets a frame image at a maximal inspiratory level or a maximal expiratory level as the reference frame image F. Further, the controller 31 acquires change amounts of a position, a distance and an angle of the rib in other respective frame images using the rib in the reference frame image F as a reference. Note that the “distance” described here is a length of a line connecting two points in a base portion among four points that equally divide each rib into three portions or a line obtained by vertically drawing a line from a point in an end portion to the rib below the point, for example, as illustrated in FIG. 10A. Further, the “angle” is an angle formed by three points of points on the both ends and a midpoint of two points of the base portion, for example as illustrated in FIG. 10B.

As described above, if the controller 31 detects an analysis portion in response to an instruction by the user and sets the ROIa, an analysis result with higher accuracy and high reproducibility can be obtained at higher speed than in a case where the user sets the ROIa.

Returning to FIG. 3, after the ROIa and the reference frame image F are set, the controller 31 tracks the set ROIa in a time direction (step S106).

In this manner, the controller 31 functions as a tracker by executing tracking processing of tracking the ROIa.

Examples of a method for tracking the ROIa set in the reference frame image F in other frame images can include, for example, template matching. In the template matching, the controller 31 sets an image region of the ROIa set in the reference frame image F as a template image. Then, the controller 31 tracks the same portion as the ROIa set in the reference frame image F in a time direction in other frame images. A method for tracking the ROIa is not limited to the template matching. In the present invention, the user can select a publicly known tracking algorithm as appropriate from channel and spatial reliability tracking (CSRT), Dasiam, minimum output sum of squared error (MOSSE), Kernelized correlation filter (KCF), Boosting, multiple instance learning (MIL), GOTURN, Median-Flow, Lucas-Kanade, RLOF, DeepFlow, and the like.

Note that the controller 31 may correct a tracking position of a specific frame image on the basis of tracking position results in a plurality of frame images. For example, in a case where a tracking position is greatly displaced in a specific frame image, the controller 31 may correct the tracking position on the basis of tracking position information of adjacent frame images.

Further, the controller 31 may set a fixed portion in accordance with the ROIa. For example, even if the subject moves his/her knee to measure motion of the knee, not only the knee, but the whole foot moves, and motion of the foot may become noise. Thus, in a case where the ROIa is set at the knee, the controller 31 sets the femur as the fixed portion. Then, the controller 31 corrects movement of the femur in each frame image assuming that there is no motion of the femur which is the fixed portion. In this manner, by the controller 31 correcting movement of an unnecessary potion other than the ROIa among the subject, an analysis result of the ROIa which is desired to be originally obtained can be favorably obtained.

Note that whether or not to automatically set the fixed portion in accordance with the ROIa is preferably changeable, for example, upon setting, or the like, of the analysis conditions in step S104.

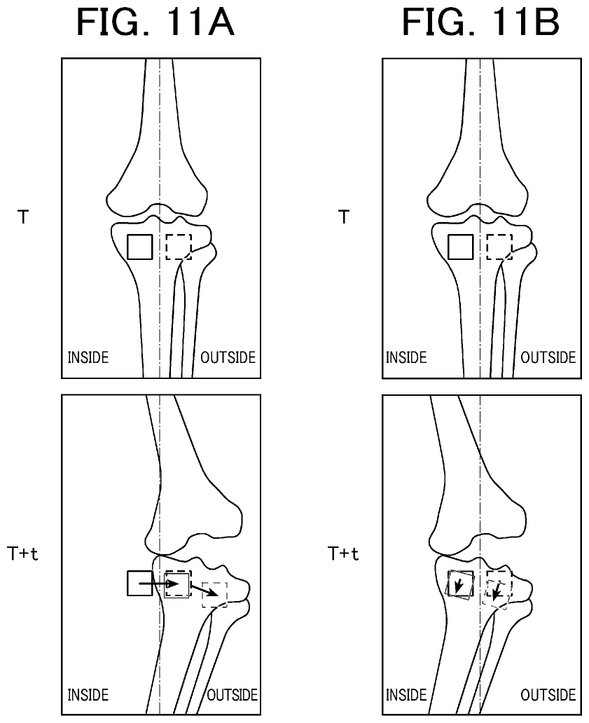

Alternatively, for example, even if a dynamic image is radiographed to measure motion of the knee, if a rotation axis of the center of the joint moves, as illustrated in FIG. 11, a viewpoint can become unstable. Thus, as illustrated in FIG. 11B, the controller 31 may position each frame image on the basis of the tracking result in one dynamic image. This can reduce troubles of adjusting a display size when the user visually confirms the analysis result.

Further, as described above, the reference frame image F in which the ROIa is set may be any frame image in the dynamic image. Thus, tracking in a “time direction” in step S106 is not limited to a forward direction. For example, in a case where the reference frame image F is the last frame image, tracking may be performed in a reverse direction. Further, in a case where the reference frame image F is neither the first frame image nor the last frame image, the direction is not limited to either the forward direction or the reverse direction, and tracking may be performed in both directions.

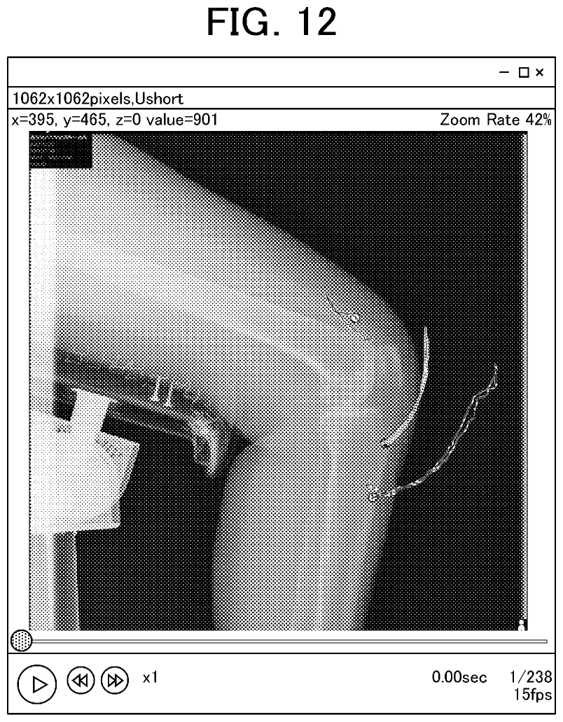

The controller 31 which has completed tracking of the ROIa outputs an image in which all tracking lines are superimposed on the dynamic image as illustrated in FIG. 12 to the display 34 (step S107). For example, in a case where the ROIa is a rectangle, the controller 31 causes, for example, a trajectory of the center point to be displayed. By displaying the image in which the tracking lines are superimposed on the dynamic image in this manner, it is possible to prevent oversight in a case where the user visually confirms only the dynamic image. Further, the user can quickly confirm the analysis result, so that diagnosis efficiency is improved.

The controller 31 functions as an output device by outputting the tracking result in this manner.

Note that in step S107, the tracking lines may be arbitrarily switchable between display and non-display through operation by the user.

Further, there is a case where the ROIa may go out of the frame, and the user may be unable to visually confirm the analysis result depending on a radiographing magnification of the dynamic image and motion of the subject. Thus, the controller 31 may lower a display magnification of the analysis result so that at least all analysis lines fall within the display 34 in accordance with the analysis result.

The controller 31 that has outputted the tracking result to the display 34 determines whether or not an external output of the tracking result is accepted (step S108). In a case where the external output of the tracking result is accepted (step S108: Yes), the controller 31 externally outputs the tracking result to the display 34 (step S109).

An arbitrary format may be used as an external output format of the tracking result by the controller 31 in step S109. For example, the controller 31 can output a file of coordinates of tracking points in each frame image as comma-separated values (CSV) data.

Alternatively, the controller 31 can externally output the tracking result, for example as a graph. The graph to be externally output is, for example, a graph which indicates time on a horizontal axis and indicates a distance between two points on a vertical axis, a graph which indicates a frame number on a horizontal axis and indicates an angle formed by three points on a vertical axis, or the like. In this manner, by causing the analysis result to be displayed as a graph, the user can quickly visually confirm the organized analysis result. This results in increasing diagnosis efficiency of the user.

By such external output data being read with the present image processing tool along with the dynamic image, a dynamic image in which the tracking result is superimposed again can be displayed at the display 34. This makes it possible to display the analysis result of the dynamic image as necessary without performing re-analysis, so that diagnosis efficiency can be improved. Further, it is not necessary to store the dynamic image including the analysis result separately from the dynamic image before analysis, so that it is possible to reduce data capacity of the radiographic imaging system 100.

Note that an external output destination of the tracking result is not limited to the display 34 and may be other external apparatuses. Particularly, the tracking result may be able to be output to the PACS.

In a case where the external output of the tracking result is not accepted (step S108: No) or after the tracking result is externally output, the controller 31 ends the dynamic image analysis processing.

### Effects of Embodiment

As described above, the image processing apparatus 3 according to the present embodiment includes an acquirer that acquires a plurality of frame images constituting a radiographed dynamic image of the subject. Further, the image processing apparatus 3 according to the present embodiment includes a setter that sets an analysis portion in a reference frame image F among the frame images. Still further, the image processing apparatus 3 according to the present embodiment includes a selector that selects one tracking algorithm from a plurality of tracking algorithms. Yet further, the image processing apparatus 3 according to the present embodiment includes a tracker that tracks the analysis portion in a time direction on the basis of the one tracking algorithm. Further, the image processing apparatus 3 according to the present embodiment includes an output device that outputs the tracking result by the tracker.

According to the configuration, it is possible to select an appropriate analysis algorithm from a plurality of analysis algorithms in accordance with an analysis target.

### Other Configurations

Note that while in the above description, the user sets the analysis conditions of one dynamic image in step S104, the present invention is not limited to this. The controller 31 may function as a selector that selects a tracking algorithm appropriate for one dynamic image. While in the above description, step S105 of setting the ROIa is executed after step S104 of setting the analysis conditions, in the present configuration, the user sets the ROIa first. Then, the controller 31 selects an appropriate tracking algorithm from information on the set ROIa.

The information on the ROIa includes, for example, information related to a shape as to whether the ROIa is an edge portion, a corner portion or neither of them. Further, the information on the ROIa includes information related to a signal to noise ratio (SN ratio). Still further, the information on the ROIa includes information related to a shape of the ROIa as to whether the ROIa is a polygon or a rectangle. Yet further, the information on the ROIa includes information on the image such as whether or not there is a structure such as a bone, a site, a frame rate, a total number of frames, a pixel size, a subject and motion of the subject.

Which information among the information on the ROIa described above is used as a basis of selecting the tracking algorithm by the controller 31 can be arbitrarily set as appropriate. For example, in a case where the frame rate is equal to or less than 7.5 fps, the controller 31 selects CSRT as the tracking algorithm. Further, for example, also in a case where the motion of the subject is large in one dynamic image, and the subject goes out of the frame, the controller 31 selects CSRT as the tracking algorithm.

Further, for example, in a case where the number of frame images constituting one dynamic image is equal to or larger than 200, the controller 31 selects KCF as the tracking algorithm. Still further, for example, also in a case where motion of an object is small between the frame images, the controller 31 selects KCF as the tracking algorithm.

Yet further, for example, in a case where a corner of an edge of the subject is set as the analysis portion, and the analysis portion is tracked in one dynamic image, the controller 31 selects a tracking algorithm based on optical flow such as Lucas-Kanade and DeepFlow.

Further, in a case where a desired analysis result cannot be obtained, it may be possible to perform re-analysis in accordance with an instruction of the user. The controller 31 accepts a re-analysis instruction from the user, for example, between step S107 and step S108. In a case where the controller 31 accepts a re-analysis instruction from the user, the controller 31 causes the processing to transition to step S106 after changing the reference frame image F, the ROIa, and the like, in accordance with the instruction of the user and tracks image characteristics again.

According to the configuration, a more accurate result can be utilized as the analysis result, which can improve diagnosis efficiency.

Note that while in the above description, the reference frame image F, the ROIa, and the like, are changed in accordance with the instruction of the user, the controller 31 may automatically change these. Further, in a case where the controller 31 accepts re-analysis, the controller 31 may function as a learner having a learning function of reflecting the result in automatic analysis from the next time.

Further, in a case where connection with an external moving image analysis workstation is detected, the controller 31 may make a list of dynamic images in the moving image analysis workstation and display the list at the display 34. Then, in a case where the user selects one dynamic image from the list, the controller 31 may perform dynamic image analysis processing using each function of the image processing tool according to the present embodiment. According to the configuration, it is not necessary to copy the dynamic image to the image processing apparatus 3 or install the image processing tool in the moving image analysis workstation. In other words, the dynamic image can be easily analyzed.

Further, the controller 31 may be able to generate a blend image obtained by blending a plurality of analysis images selected by the user at an arbitrary ratio. According to the configuration, the user can visually confirm two analysis images at one display 34. Further, the user can comprehensively give a diagnosis on the basis of information obtained from a plurality of different analysis methods.

Note that the blend image is not limited to an image including two analysis images and may be an image including three or more images.

While in the above description, an example has been described where a non-volatile semiconductor memory and a hard disk are used as computer readable media of the program according to the present invention, the present invention is not limited to this example. As other computer readable media, a read only memory (ROM) which is a volatile memory, and a portable recording medium such as a CD-ROM can be applied. Further, carrier waves can be also applied to the present invention as a medium of providing data of the program according to the present invention via a communication line.

Although embodiments of the present invention have been described and illustrated in detail, the disclosed embodiments are made for purposes of illustration and example only and not limitation. The scope of the present invention should be interpreted by terms of the appended claims.

What is claimed is:

1. An image processing apparatus comprising a hardware processor that:
acquires a plurality of frame images constituting a radiographed dynamic image of a subject;
sets an analysis portion in a reference frame image among the plurality of frame images;
selects one tracking algorithm from a plurality of tracking algorithms;
tracks the analysis portion in a time direction on a basis of the one tracking algorithm; and
outputs a tracking result of the tracking.

2. The image processing apparatus according to claim 1, wherein the hardware processor selects the one tracking algorithm from the plurality of tracking algorithms on a basis of selection by a user.

3. The image processing apparatus according to claim 1, wherein the hardware processor selects the one tracking algorithm from the plurality of tracking algorithms on a basis of information on the analysis portion.

4. The image processing apparatus according to claim 1, wherein the hardware processor selects spatial reliability tracking (CSRT) as the one tracking algorithm in a case where a frame rate of the plurality of frame images is equal to or less than 7.5 fps or in a case where frame-out occurs in one of the plurality of frame images.

5. The image processing apparatus according to claim 1, wherein the hardware processor selects Kernelized correlation filter (KCF) as the one tracking algorithm in a case where a number of frames of the plurality of frame images is equal to or larger than 200.

6. The image processing apparatus according to claim 1, wherein the hardware processor selects a tracking algorithm based on optical flow as the one tracking algorithm in a case where a corner of an edge of the subject is set as the analysis portion.

7. The image processing apparatus according to claim 1, further comprising:
a display that displays the tracking result.

8. An image processing method of an image processing apparatus, comprising:
inputting a plurality of frame images constituting a radiographed dynamic image of a subject;
setting an analysis portion in a reference frame image among the frame images;
selecting one tracking algorithm from a plurality of tracking algorithms;
tracking the analysis portion in a time direction on a basis of the one tracking algorithm; and
outputting a tracking result of the tracking.

9. A non-transitory computer readable recording medium storing a program for causing a computer of an image processing apparatus to perform:
acquiring a plurality of frame images constituting a radiographed dynamic image of a subject;
setting an analysis portion in a reference frame image among the plurality of frame images;
selecting one tracking algorithm from a plurality of tracking algorithms;
tracking the analysis portion in a time direction on a basis of the one tracking algorithm; and
outputting a tracking result of the tracking.
